(19) United States
(12) Patent Application Publication
(10) Pub. No.: US 2018/0224395 A1
(43) Pub. Date: Aug. 9, 2018

(54) METHODS, APPARATUSES, AND SYSTEMS FOR STABILIZING NANO-ELECTRONIC DEVICES IN CONTACT WITH SOLUTIONS

(71) Applicant: Arizona Board of Regents on behalf of Arizona State University, Scottsdale, AZ (US)

(72) Inventors: Stuart LINDSAY, Phoenix, AZ (US); Pei PANG, Tempe, AZ (US)

(21) Appl. No.: 15/946,873

(22) Filed: Apr. 6, 2018

Related U.S. Application Data
(63) Continuation of application No. 15/121,148, filed on Aug. 24, 2016.
(60) Provisional application No. 61/944,322, filed on Feb. 25, 2014.

(51) Int. Cl. G01N 27/416; G01N 27/403; G01N 27/30; G01N 27/48; C12Q 1/6869; G01N 33/487

(52) U.S. Cl. CPC G01N 27/4163; G01N 27/403; G01N 27/301; G01N 27/48; C12Q 2563/157; C12Q 1/6869; G01N 33/48721; C12Q 2563/116

(57) ABSTRACT

An apparatus for identifying and/or sequencing one or more molecules including a first sensing electrode and a second sensing electrode separated from the first electrode by a gap. An electrolyte is contained within the gap. The surfaces of the first sensing electrode and the second sensing electrode are functionalized with adaptor molecules for contacting the one or more molecules. The apparatus also includes a reference electrode in contact with the electrolyte and coupled to one of the sensing electrodes.

### CROSS-REFERENCE TO RELATED APPLICATIONS

This application is a continuation application of U.S. application Ser. No. 15/121,148, filed Aug. 24, 2016, which is a U.S. national phase of PCT/US2015/017519, filed Feb. 25, 2015, and claims priority to U.S. Provisional Application No. 61/944,322 titled “METHODS, APPARATUSES AND SYSTEMS FOR STABILIZING NANO-ELECTRIC DEVICES IN CONTACT WITH SOLUTIONS”, filed Feb. 25, 2014, the entire disclosure of which is incorporated herein by reference in its entirety.

### STATEMENT OF FEDERALLY-SPONSORED RESEARCH

This invention was made with government support under R01 HG006323 awarded by the National Institutes of Health. The government has certain rights in the invention.

### BACKGROUND

A nanoscale electronic device for detecting and analyzing single molecules based on recognition tunneling (RT) has been described previously (see, e.g., U.S. patent application publication no. 2014/0113386), which uses a one Palladium (Pd) electrode having a layer of Al₂O₃ (insulator). Another electrode is included which has a Pd layer deposited on top of an insulating layer. An opening or gap is established through the layers and the exposed metal functionalized with adaptor molecules serve to trap analytes in a well-defined chemical configuration. An example of an adaptor molecule is 4(5)-(2-mercaptoethyl)-1H-imidazole-2-carboxamide, hereafter referred to as ICA. Upon a voltage being applied across the gap, a series of current spikes are generated upon which are based on molecules (e.g., analytes) which pass through the gap and bridge one electrode to the other via adaptor molecules functionalized on the electrodes. The current spikes are analyzed (e.g., via a machine learning algorithm) to identify the particular analyte within in the gap for an associated current spike.

However, charged analyte molecules that adsorb onto the electrodes can change the potential of the electrode, which can cause a significant effect on the current spikes produced, due to the relatively small scale of the RT apparatus. This problem has been recognized, for example, in apparatuses made of a single semiconductor nanowire (Xie, P., Q. Xiong, Y. Fang, Q. Qing, and C. M. Lieber, Local Electrical Potential Detection of DNA by Nanowire-Nanopore Sensors. Nature Nanotechnology, 2012. 7: p. 119-125) or a single carbon nanotube (Sims, P. C., I. S. Moody, Y. Choi, C. Dong, M. Iftikhar, B. L. Corso, O. T. Gul, P. G. Collins, and G. A. Weiss, Electronic Measurements of Single-Molecule Catalysis by Camp-Dependent Protein Kinase A. J. Am Chem Soc, 2013. 135: p. 7861-7868), and an attempt at stabilizing an RT apparatus by connecting it to a reference electrode 24 in contact with the solution (see, e.g., apparatus 21 (FIG. 2). Such an arrangement may be used to maintain the surface of the apparatus at the same potential as the reference electrode, despite adsorptions of charged molecules that may occur. In such nanowire devices, only small biases V (22) are applied across the length of the entire device.

In some RT apparatuses, the problem may be more complex since a significant bias voltage V is applied across a relatively small gap in contact with the solution. Bias V can be on the order of about 0.5V, and thus, if one electrode is at a potential where interactions with the solution are small, the other electrode may not be, which can cause instability in the RT junction. FIG. 3a (where the analyte comprises the nucleotide dAMP) and FIG. 3b (where the analyte comprises the nucleotide dGMP) illustrate swings in current output with slow returns to the baseline current (see arrows), which is understood not to be associated with a tunneling process, but rather by relatively slow, i.e., on the order of a number of seconds, adsorptions of charged species and release thereof from the solution in contact with the electrodes. Additionally, RT apparatuses may become inactive after only a few minutes of operation. Accordingly, it is desirable to find a way to stabilize a multiple (e.g., two) electrode sensing device in contact with a conducting solution.

### SUMMARY

An apparatus for identifying and/or sequencing one or more first molecules includes a first sensing electrode and a second sensing electrode separated from the first electrode. The apparatus further includes a gap established by the separated electrodes, wherein an electrolyte is contained within the gap. The electrode surfaces are functionalized with adaptor molecules for contacting one or more first molecules. The apparatus further includes a reference electrode in contact with the electrolyte and coupled to one of the sensing electrodes.

### BRIEF DESCRIPTION OF THE FIGURES

FIG. 1 illustrates an RT apparatus and reference electrode according to some embodiments, whereby separate solution compartments are provided above and below the device (the corresponding solutions therein may be labeled cis solution and trans solution). V is the bias applied between top and bottom electrodes and V_{ref} is the bias applied with respect to a reference electrode.

FIG. 2 illustrates the use of a reference electrode with nanowire devices, according to the prior art.

FIGS. 3a-3d illustrates current spike results of an RT apparatus which lack a reference electrode (3a and 3b), and yield unstable current outputs, and current spike results of an RT apparatus which includes a reference electrode (3c and 3d) which are stable and operate for long periods.

FIG. 4 illustrates current-voltage sweeps of an imidazole (ICA) coated Pd electrode showing the large currents that develop as a consequence of hydrogen evolution when the potential is swept negative of 0V with respect to a Ag/AgCl reference electrode.

FIG. 5a shows cyclic voltammetry for an ICA coated Pd electrode from +50 mV with respect to a Ag/AgCl reference electrode. The system is stable against hydrogen evolution, but now shows electrochemical noise that peaks at +380 mV. FIG. 5b illustrates that a bare Pd electrode does not display the electrochemical noise of FIG. 5a. FIG. 5c shows RT signals from a junction in which the lower electrode is held at +100 mV vs. Ag/AgCl. Noise spikes are evident starting around 280 mV, corresponding to +380 mV as Ag/AgCl.

### DETAILED DESCRIPTION

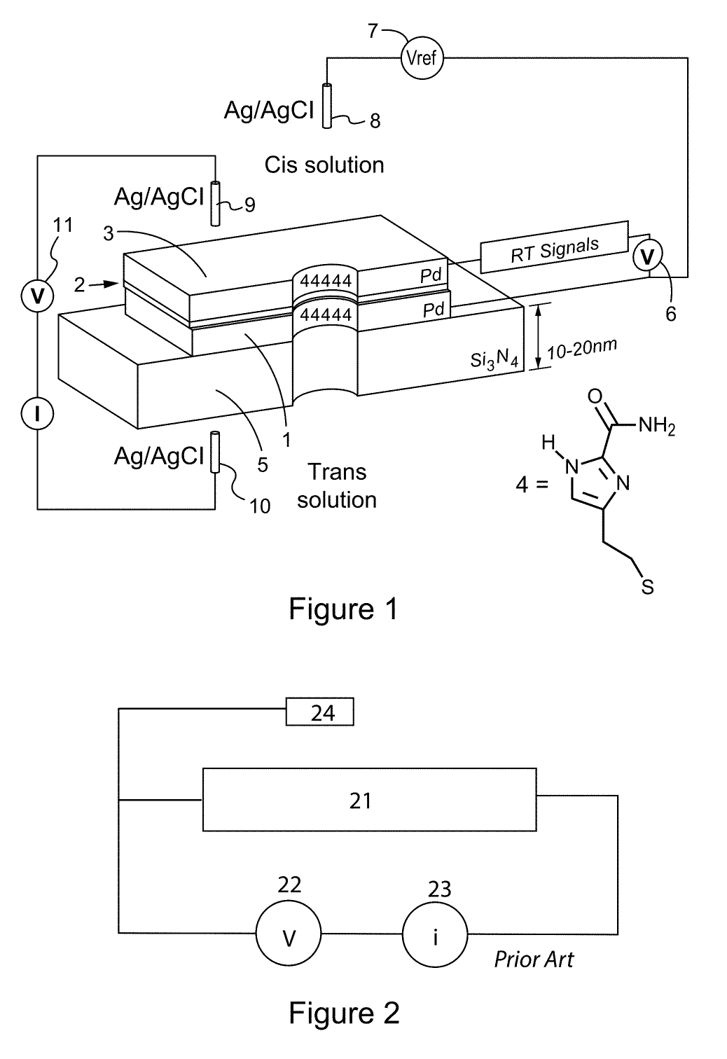

As shown in FIG. 1, a first electrode having a layer of Pd (for example) of about 10 nm thickness 1 onto which a layer of an insulator 2 of Al₂O₃ (for example) of about 2 nm thick, has been deposited. A second electrode 3, which may also include a layer of Pd, for example, of about 10 nm thick) deposited on top of the insulating layer. An opening/gap is established through the layers and the exposed metal functionalized with adaptor molecules (e.g., ICA) 4 serve to trap analytes in a well-defined chemical configuration. Upon a voltage V (6) being applied across the gap, a series of current spikes are generated upon which are based on molecules (e.g., analytes) which pass from one electrode to another via the functionalized adaptor molecules and the trapped analytes.

In some embodiments, an RT apparatus includes a reference electrode 8, comprising, for example, a silver wire covered in a silver chloride layer, which is placed in contact with the solution and connected to either one of the electrodes via a voltage source Vref 7, where Vref is selected to maximize the stability of the two-electrode device operated at a bias V 6. The reference can be connected to either one of the electrodes in the RT device, so long as the other electrode is held at a fixed potential difference with respect to the electrode that is connected to the reference electrode. In some embodiments, the criteria for setting the value of Vref for stable operation are as described below. In some embodiments, reference electrodes 9, 10 can be placed in contact with solution above and (and/or) below the tunnel junction with a second bias 11, which may be applied to drive charged molecules through the tunnel junction (if desired).

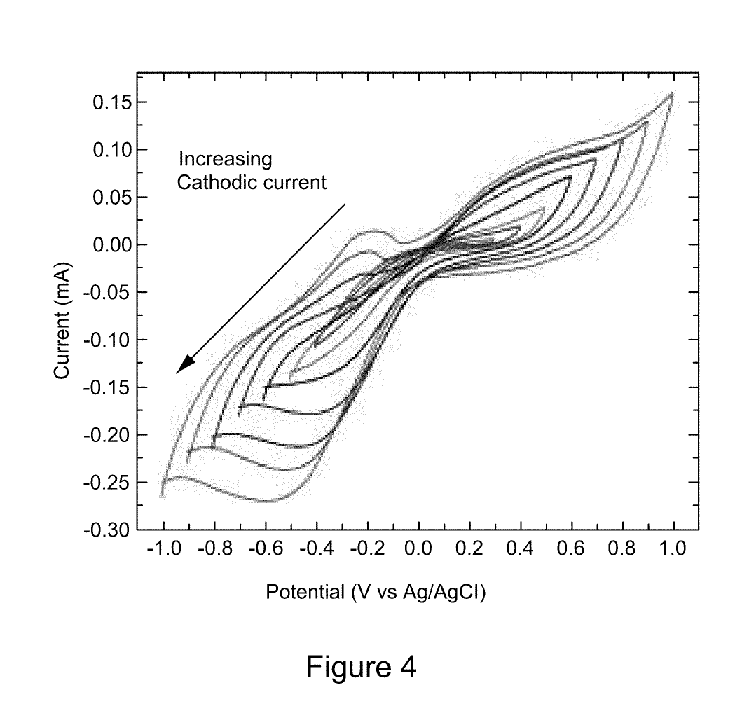

In some embodiments, electrochemical data is acquired to aid in selecting values for Vref 7, and/or V, the bias across the apparatus 6. FIG. 4 illustrates a series of cyclic voltammograms obtained using a Pd electrode coated with a monolayer of ICA. In these sweeps, the potential range of the sweep was increased in steps around 0 V vs. Ag/AgCl. Large currents are generated at the electrode is swept further negative of 0 V, a consequence of hydrogen evolution (Burke, L. D. and J. K. Casey, An Examination of the Behavoir of Palladium Electrodes in Acid. J. Electrochem. Soc., 1993. 104: p. 1284-1291). In consequence, a device that adsorbs negatively charged molecules can be driven into the potential range where hydrogen is evolved, destabilizing the device. In some embodiments, Vref is chosen such that each electrode is not at a potential where electrochemical reactions occur with the molecules or ions in the solution in contact with the electrodes.

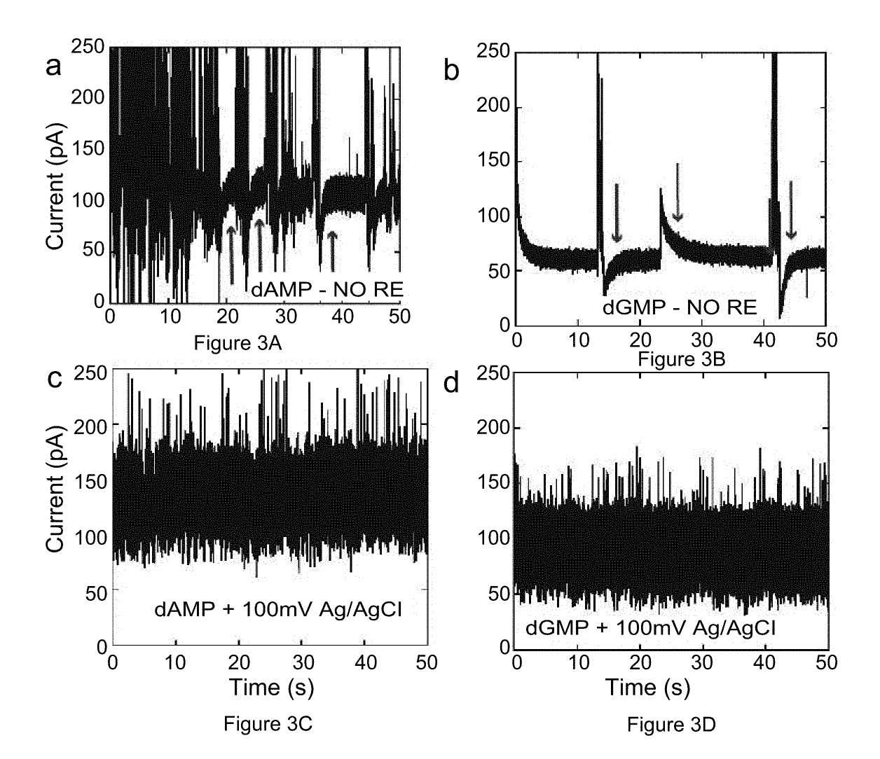

An example of this instability is shown in FIGS. 3a and 3b. This shows RT signals obtained in the presence of 100 micromolar adenosine monophosphate (FIGS. 3a) and 100 micromolar guanosine monophosphate (FIG. 3b). The signal spikes generated by recognition tunneling occur in bursts but are accompanied b violent current fluctuations with large changes in the background current (pointed to by arrows). In the case of dGMP (FIG. 3b) the device generates RT signals for only a small fraction of the time. After a few minutes of operation, the devices always became inactive.

The stability of the apparatus may be improved by apparatuses and methods disclosed herein. FIG. 3c (dAMP) and FIG. 3d (dGMP) show how violent current fluctuations may be removed, the normal recognition-tunneling signal restored, and the baseline current stabilized, when V_{ref} is set to about +100 mV (bottom electrode with respect to Ag/AgCl). Such stabilized apparatuses have operated continuously for periods of 10 h or more. In this particular example, V_{ref} was chosen so that the electrode connected to the reference was still slightly positive of the potential for hydrogen evolution (which is about −150 mV on the Ag/AgCl scale). In turn, the second electrode was held at a potential, V_{ref}+V_{bias} that is less than the potential for oxidation reactions to occur in this solution. Thus both electrodes are held at potentials such that electrochemical reactions are avoided.

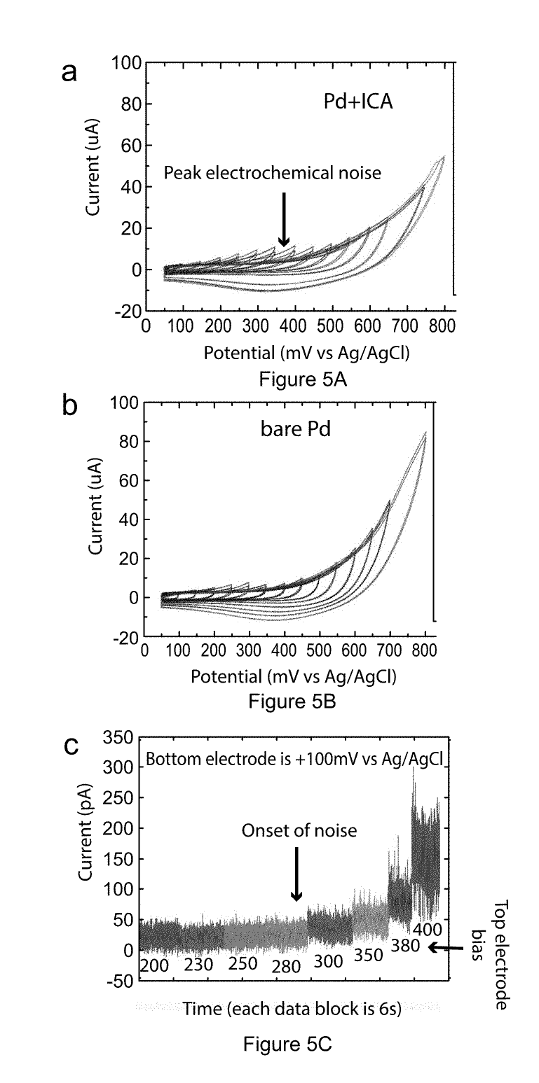

The second electrode (3 in FIG. 1) is held at a potential V_{ref}+V with respect to the Ag/AgCl reference (8 in FIG. 1). Its electrochemical stability is also important. As an example, FIG. 5a shows cyclic voltammetry of a Pd electrode functionalized with an ICA monolayer. The sweeps start at +50 mV vs. Ag/AgCl and the upper amplitude is increased in steps up to 750 mV. For comparison, FIG. 5b shows cyclic voltammetry on bare Pd. The increase of current at the highest bias clearly reflects an oxidation process on the Pd surface (suppressed somewhat when the Pd is covered with ICA because the currents are lower—FIG. 5a). However, the ICA covered surface also shows evidence of some excess current peaking around 380 mV. FIG. 5c shows RT signals obtained from a tunnel junction with the lower electrode (1 in FIG. 1) held at +100 mV vs Ag/AgCl. Extra noise spikes occur when the bias applied to the top electrode (3 in FIG. 1) exceeds about 280 mV. Thus, these new features are associated with the electrochemical signals observed at about +380 mV with respect to Ag/AgCl. Accordingly, an optimal operating point for this device, in this example, is to have one electrode held at +100 mV vs. Ag/AgCl while the second electrode should not exceed +350 mV vs Ag/AgCl. A device operated in these conditions gives excellent chemical recognition signals, is stable, and substantially free of additional noise for long periods. Without the reference electrode connected as described, the device becomes noisy with large shifts in baseline, as illustrated in FIGS. 3a and 3b.

In some embodiments, additional improvements may be made by including a thick polymer layer, which may be deposited by spin coating of PMMA resist, with an opening above the junction which may be used as both a mask, to cut the opening through the electrodes, as well as a fluid well to keep solutions from the electrodes (except in the vicinity of the tunnel junction). Accordingly, for such embodiments, this process may eliminate leakage currents when the solution (which is contacting the biased reference electrode) also made a large contact area with the tunneling apparatus by virtue of solution leakage over the surface of the apparatus. To that end, electrodes can be cut using, for example, reactive ion etching, with Cl gas used to tech the Pd electrodes and BCl₃ gas used to etch the Al₂O₃.

In some embodiments, the reference electrode may comprise an Ag wire coated with AgCl salt, although one of skill in the art will appreciate that any electrode of substantially constant polarization will suffice. Non-limiting examples of such electrodes include the silver/silver chloride electrode, the saturated calomel electrode, the normal hydrogen electrode, and/or the like. Even a bare silver, palladium or platinum wire will do so long as its area is many thousands of times as large as the area of the tunneling elecrtodes exposed to the electrolyte so that its potential only changes by a small amount when ions and molecules absorb or desorb form its surface. Accordingly, any large metallic electrode (in some embodiments, much larger than the sensing electrodes 1 and 3 in FIG. 1) may suffice so long as it is sized so as to undergo small changes, i.e., less than a few tens of mV, in potential as charged species are absorbed and/or desorbed from its surface. In this manner, a reference electrode can be built into a device by fabricating a large (e.g., at least a micron by a micron in area) metal pad in a position such that it is in contact with the electrolyte.

In some embodiments, an apparatus for identifying and/or sequencing one or more first molecules is provided, and comprises a first sensing electrode, a second sensing electrode separated from the first electrode, and a gap established by the separated electrodes. An electrolyte is contained within the gap and the electrode surfaces are functionalized with adaptor molecules for contacting one or more first molecules. The apparatus also includes a reference electrode in contact with the electrolyte and coupled to one of the sensing electrodes. In some such embodiments, the apparatus may further comprise a voltage source for coupling the reference electrode with one of the sensing electrodes, where the voltage source is configured to hold the sensing electrode coupled to the reference electrode at a constant potential difference with respect to the reference electrode.

In some embodiments, a method determining the potential of a reference electrode in a recognition tunneling (RT) apparatus is provided. The RT apparatus may comprise a first sensing electrode, a second sensing electrode separated from the first electrode, and a gap established by the separated electrodes. An electrolyte is contained within the gap, and the electrode surfaces are functionalized with adaptor molecules for contacting one or more first molecules. The apparatus may further comprise a reference electrode in contact with the electrolyte and coupled to one of the sensing electrodes, and a voltage source for coupling the reference electrode with the first sensing electrode. The voltage source is configured to hold the first sensing electrode at a constant potential difference with respect to the reference electrode. The method comprises sweeping the bias between the first sensing electrode and the reference electrode, recording a leakage current through the first sensing electrode, and the noise for each of a plurality of fixed values of potential difference between first sensing electrode and the reference electrode, and selecting the reference electrode potential corresponding to the minimum leakage current.

Any and all references to publications or other documents, including but not limited to, patents, patent applications, articles, webpages, books, etc., presented in the present application, are herein incorporated by reference in their entirety.

Although a few variations have been described in detail above, other modifications are possible. For example, any logic flow or arrangement of elements/structure depicted in any figure and/or described herein does not require the particular order/arrangement shown, or sequential order, to achieve desirable results. Other implementations may be within the scope of at least some of the following example claims which follow.

As noted elsewhere, the disclosed embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include formulations, methods, systems and devices which may further include any and all elements from any other disclosed formulations, methods, systems, and devices, including any and all elements corresponding to RT systems. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Finally, some embodiments disclosed herein may be distinguishable over prior art because, in some such embodiments, one and/or another element/step/structure included with prior art formulations, methods, systems and devices are missing from some of the embodiments disclosed herein, and as a result of such missing elements, such embodiments are patentable over the prior art.

What is claimed is:

1. A method for stabilizing a recognition tunneling device, wherein the recognition tunneling device comprises: (a) a first sensing electrode, (b) a second sensing electrode separated from the first sensing electrode by a gap, (c) an electrolyte in contact with the first and second sensing electrodes, and (d) a reference electrode of essentially constant polarization, wherein the reference electrode is in contact with the electrolyte and coupled to the first sensing electrode,
the method comprising:
(i) determining a potential range over which the first sensing electrode does not generate a Faradaic current in the electrolyte, the potential range having a lower bound and an upper bound, wherein the potential range is determined relative to the reference electrode;
(ii) holding the first sensing electrode at a potential no less than the lower bound with respect to the reference electrode; and
(iii) holding the second sensing electrode at a potential no more than the upper bound with respect to the first sensing electrode.

2. The method of claim 1, wherein the first and second sensing electrodes are functionalized with an adaptor molecule.

3. The method of claim 2, wherein the adaptor molecule is 4-(2-mercaptoethyl)-3λ²-imidazole-2-carboxamide.

4. The method of claim 1, wherein the reference electrode is selected from the group consisting of a silver/silver chloride electrode, a saturated calomel electrode, and a normal hydrogen electrode.

5. The method of claim 4, wherein the reference electrode is a silver/silver chloride electrode.

6. The method of claim 1, wherein the reference electrode is configured to change by less than a few tens of mV in potential as charged species from the electrolyte are absorbed or desorbed from its surface.

7. A method for stabilizing a recognition tunneling device, wherein the recognition tunneling device comprises: (a) a first sensing electrode, (b) a second sensing electrode separated from the first sensing electrode by a gap, (c) an electrolyte in contact with the first and second sensing electrodes, and (d) a reference electrode of essentially constant polarization, wherein the reference electrode is in contact with the electrolyte and coupled to the first sensing electrode,
the method comprising:
(i) determining a potential range over which the first sensing electrode does not generate a Faradaic current in the electrolyte, the potential range having a lower bound and an upper bound, wherein the potential range is determined relative to the reference electrode;
(ii) holding the first sensing electrode at a potential no more than the upper bound with respect to the reference electrode; and
(iii) holding the second sensing electrode at a potential no less than the lower bound with respect to the first sensing electrode.

8. The method of claim 7, wherein the first and second sensing electrodes are functionalized with an adaptor molecule.

9. The method of claim 8, wherein the adaptor molecule is 4-(2-mercaptoethyl)-3λ²-imidazole-2-carboxamide.

10. The method of claim 7, wherein the reference electrode is selected from the group consisting of a silver/silver chloride electrode, a saturated calomel electrode, and a normal hydrogen electrode.

11. The method of claim 10, wherein the reference electrode is a silver/silver chloride electrode.

12. The method of claim 7, wherein the reference electrode is configured to change by less than a few tens of mV in potential as charged species from the electrolyte are absorbed or desorbed from its surface.
